(19) United States
(12) Patent Application Publication
(10) Pub. No.: US 2007/0020696 A1
(43) Pub. Date: Jan. 25, 2007

(54) Appl proteins as rab5 effectors

(76) Inventors: Marino Zerial, Dresden (DE); Marta Miaczynska, Krakow (PL)

Correspondence Address: FULBRIGHT & JAWORSKI L.L.P., 600 CONGRESS AVE., SUITE 2400, AUSTIN, TX, 78701, US

(21) Appl. No.: 10/564,435

(22) PCT Filed: Jul. 8, 2004
(86) PCT No.: PCT/EP04/07527

§ 371 Date: Aug. 30, 2006

(30) Foreign Application Priority Data
Jul. 10, 2003 (EP) 03015788.7

(51) Int. Cl. G01N 33/567; G01N 33/53

(52) U.S. Cl. 435/7.2

(57) ABSTRACT

The present invention relates to an in vivo-assay to screen for anti-proliferative drugs, the assay comprising the steps of: (a) contacting cells of a primary cell culture or of an established cell line with a candidate substance, (b) subsequently or concomitantly with a candidate substance, contacting the cells with a growth factor, (c) processing the cells for immunofluorescence staining to detect APPL1 and APPL2 using an anti-APPL1 and/or 2 antibody, or alternatively using GFP-tagged APPL proteins stably or transiently expressed by the cells via transfection, (d) assessing the degree of colocalisation of APPL1 and/or 2 and the growth factor, the solubilisation of APPL1 and/or 2 and their translocation to the nucleus, (e) repeating steps (b) to (d) with cells not previously treated with the candidate substance, and (f) comparing the degree of colocalisation of APPL1 and/or 2 and the growth factor, the solubilisation of APPL1 and/or 2 and their translocation to the nucleus between the cells not previously treated with the candidate substance (untreated cells) and cells treated with the candidate substance (treated cells), wherein an altered degree of colocalisation of APPL1 and/or 2 and the growth factor, an altered solubilisation of APPL1 and/or 2 and/or their altered translocation to the nucleus in the treated vs. the untreated cells identifies the candidate substance as an anti-proliferative drug.

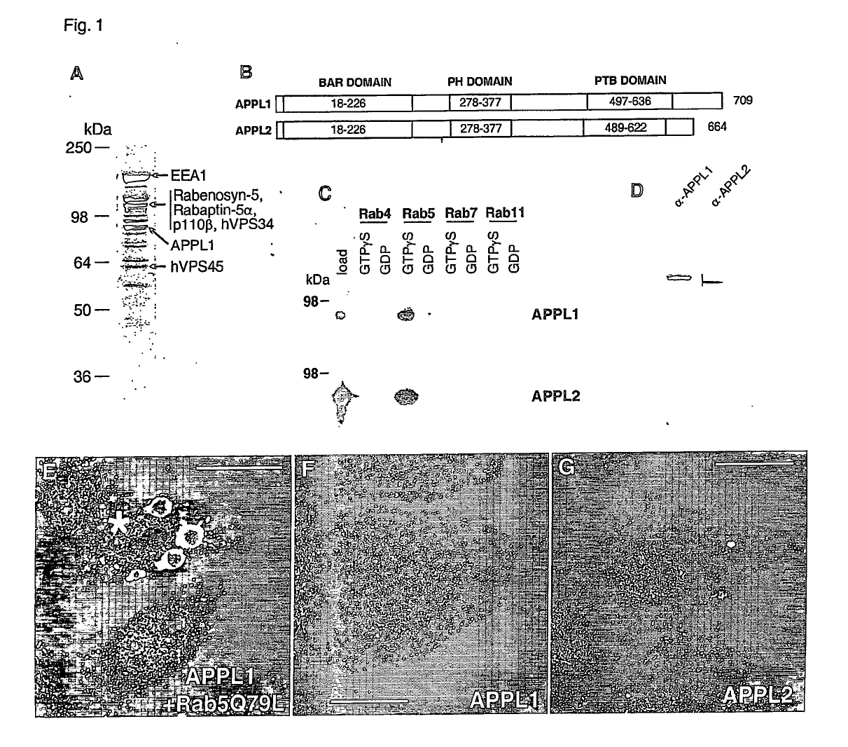

The present invention relates to the field of signal transduction. Generally, signals generated in response to extracellular stimuli at the plasma membrane are transmitted through cytoplasmic transduction cascades to the nucleus. Endocytic organelles play a role in the termination of signals but it had remained unclear whether they are also required for signal propagation. The inventors of the present invention have identified a novel vesicular structure or endocytic organelle, the hermesome, which is selectively accessible to EGF but poorly to transferrin or fluid phase markers. Hermesomes harbour APPL1 and APPL2, two novel effectors of the small GTPase Rab5, which has been known to be a key regulator of endocytosis. APPL (Adaptor protein containing PH domain, PTB domain and Leucine zipper motif; accession number AF169797; FIG. 1B), a 709 amino acid protein was previously identified in a two-hybrid screen as an interacting partner of the serine/threonine kinase AKT2/PKBβ and putative adaptor tethering inactive AKT2 to cytoplasmic PI(3)K p110α³⁰. Another two-hybrid screen described APPL (therein referred to as DIP13α) as an interactor of the tumour suppressor DCC (deleted in colorectal cancer) and a mediator of DCC-induced apoptotic signalling³¹. The inventors further identified a related Rab5 effector, a protein of 664 amino acids and 54% identity to APPL (recently named DIP13β, accession no. NM_{—}018171). The inventors refer to the two proteins as APPL1 and APPL2. In response to extracellular stimuli such as EGF and oxidative stress, APPL1 translocates from hermesomes to the nucleus. In the nucleus, APPL proteins interact with the nucleosome remodelling and histone deacetylase multi-protein complex NuRD/MeCP1, an established regulator of chromatin structure and gene expression. Both APPL1 and APPL2 are essential for cell proliferation and their function requires Rab5 binding. Thus, the inventors identified a novel pathway directly linking Rab5 to signal transduction and mitogenesis. Hermesomes are likely to have a widespread function in the form of specialized endosomes acting as intermediates in signalling between the plasma membrane and the nucleus.

In response to extracellular stimuli cells activate an intricate network of signalling cascades^{1,2}. In the traditional view, signal transduction is initiated at the plasma membrane and, via a series of protein-protein interactions and kinase cascades, transmitted through the cytoplasm to the nucleus where gene expression is modulated. In this model, endocytosis is considered merely as a mechanism for signal termination by downregulation of receptors activated at the plasma membrane and their degradation in the lysosomes. The idea that endosomes can perform a signalling function received support by studies of NGF action in neurons³. More recently, an increasing number of proteins have been shown to form structurally and functionally distinct signalling complexes with activated receptors along their intracellular itinerary through various endocytic compartments⁴⁻⁷. These findings suggest that trafficking through endosomes may play a more active role in the initiation, propagation and termination of signals than previously anticipated. To which extent, however, endosomes participate in the signal transduction process remains to be established also in view of other studies arguing against such a role⁸⁻¹⁰. It is intuitive that, owing to the essential function of endosomes in cellular homeostasis¹¹, discriminating between their role in receptor trafficking and signalling may prove a difficult task. On the other hand, there is compelling evidence that signalling pathways can modulate the endocytosis machinery⁵⁻⁷, as exemplified by the recently uncovered functional connections between the small GTPase Rab5 and signalling molecules¹²⁻¹⁴. Rab5 is a key regulator of transport from the plasma membrane to the early endosomes. Continuous cycles of GDP/GTP exchange and hydrolysis regulate the kinetics of constitutive endocytosis¹⁵ but this nucleotide cycle can also be modulated by extracellular stimuli. Stimulation by EGF enhances the rate of endocytic membrane flow¹² by increasing the fraction of active Rab5. This occurs through stimulation of the Rab5 guanine nucleotide exchange factor (GEF) RINI¹³ and downregulation of the GTPase-activating protein (GAP) RN-tre¹⁴. Beside regulating receptor intemalisation¹⁴, RN-tre is also integrated into the EGF signalling pathway via its interactions with the EGF receptor (EGFR) substrate Eps8 and the adaptor protein Grb2, which links EGFR to mSos, a GEF for Ras^{14,16}.

The molecular principles underlying the structural and functional organisation of early endosomes are also intimately linked to the function of signalling molecules. On the early endosomes, Rab5 regulates the membrane recruitment and activity of a wide range of downstream effectors¹⁷⁻¹⁹, such as Rabaptin-5α/5β/Rabex-5, EEA1, Rabenosyn-5/hVPS45 and phosphatidylinositol-3 kinases (PI(3)Ks) p110β/p85α and hVPS34/p150, which act cooperatively in vesicle tethering, SNARE priming, and endosome motility along microtubules²⁰⁻²³. Based on these data, Rab5 has been proposed to organise a domain on the early endosomes which is enriched in phosphatidylinositol 3-phosphate (PI(3)P) and a set of PI(3)P-binding effectors^{21,24}. The same phosphoinositide species is also required for the endosomal localisation of various signalling molecules, such as a component of the TGF-β pathway SARA (Smad anchor for receptor activation)^{25,26} and hepatocyte growth factor-regulated tyrosine kinase substrate, Hrs^{27,28}. Intriguingly, dominant-negative mutant of Rab5 affects TGF-β/activin signal transduction in endothelial cells by an as yet unknown mechanism²⁹.

While these examples suggested a link between Rab5 and intracellular signalling, it remained open whether components of the Rab5 machinery and the endocytic organelles harbouring them are required for signal transduction. Furthermore, whether endosomes are the only organelles involved in signal transduction or whether specialised compartments devoted to signalling exist were open questions. In identifying a novel cellular structure that does not act as housekeeping endosome but is specialized in transport of molecules involved in signal transduction and in transducing signals between the plasma membrane and the nucleus the inventors have provided an answer to these questions. Accordingly, it is an object of the present invention to describe this novel signal transduction pathway involving Rab5 and the APPL proteins as Rab5 effectors. Furthermore, this invention allows to predict the existence of other novel signalling pathways converging on the hermesome.

Thus, the gist of the present invention is to have identified two previously uncharacterised Rab5 effectors (APPL1 and 2) and uncovered a novel signalling pathway. When studying the novel pathway in some greater detail, the present inventors were able to comprehend some basic mechanisms of signal transduction and subsequently to identify a novel cellular organelle involved in signal transduction. They called the novel organelle a hermesome, which is a type of endocytic vesicle and/or endosome and exhibits on its surface both APPL1 and APPL2 and Rab5. The hermesome is involved in the propagation of signals from the cell surface to the nucleus.

The inventors propose to apply the knowledge derived from the discovery of the Rab5-APPL signalling pathway involving the hermesome to the development of new drugs to combat tumour cells and/or to induce apoptosis in tumour cells. The new strategy exploits the use of tools to monitor the endocytic and signalling pathways intersecting the hermesomes and identify chemical compounds able to modulate them. The novelty of the invention relies on the fact that such signalling pathways have never been described before and entail a new endocytic structure/organelle distinct from the canonical early endosomes.

In other words, in the course of elucidating the mechanisms of signal transduction involving hermesomes, the inventors have been able to provide some technical tools to screen for compounds/factors useful as anti-proliferative drugs to combat tumour cells and/or to induce apoptosis in tumour cells.

Thus, a first aspect of the present invention is an in vivo-assay (in vivo does not mean that it is carried out on a living animal but requires a cell culture only) to screen for anti-proliferative drugs which may be used in the manufacture of a pharmaceutical to treat cancer/tumour diseases (by combating cancer/tumour cells and/or inducing apoptosis in such cells). Based on the findings they had made previously, the inventors were able to provide such assay on the basis of various mechanisms, implying a number of different approaches for use in the screening of anti-proliferative drugs.

In detail, the present inventors have developed a method to isolate hermesomes from a cell and, subsequently, they have further developed an in vitro-assay (in vitro means that the assay is carried out by means of cell extracts rather than intact cells of a cell culture) to screen for anti-proliferative drugs. In other words, the in vitro-assay according to the invention requires previous isolation of hermesomes.

Briefly, both the in vivo- and the in vitro-assay may be based on the capability of a candidate compound (i) to interfere with the interaction between APPL1 and/or 2 and Rab5 and/or the hermesome (that is, to stabilise/destabilise the binding of APPL1 and/or 2 to Rab5 and/or the hermesome, thereby controlling the release of APPL1 and/or 2 from Rab5 and/or the hermesome into the cytoplasm); (ii) to interfere with the transport of APPL1 and/or 2 into the nucleus; (iii) to modulate the sorting and routing of growth factor receptors to hermesomes vs. endosomes; (iv) to modulate the nucleotide cycle of Rab5 specifically or primarily on hermesomes vs. endosomes, preferably by increasing the level of GTP-bound Rab5 on hermesomes; (v) to modulate, in particular prevent cytoplasmic interactions with other factors; and (vi) to modulate, in particular prevent the association of APPL1 and/or 2 with the NuRD/MeCP1 complex or its associated factors such as p53. Compounds that stabilise the binding of APPL1 and/or 2 to Rab5and/or the hermesome prevent the transport of APPL into the nucleus, prevent the sorting and routing of growth factor receptors to hermesomes vs. endosomes.

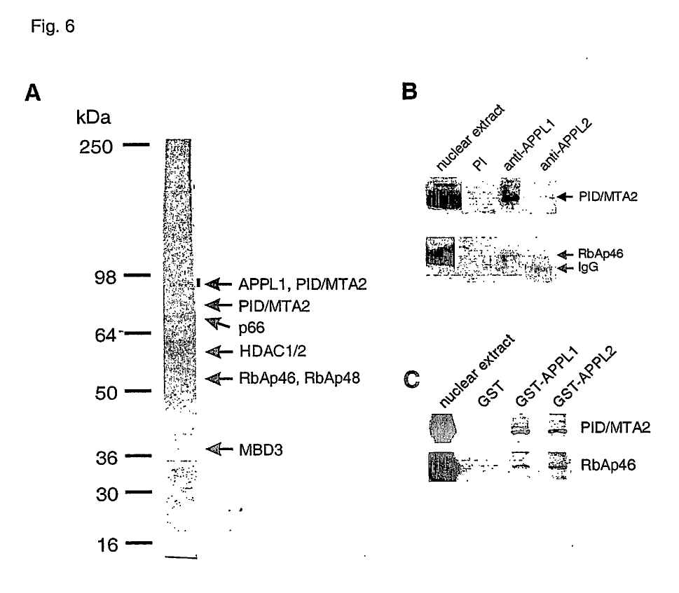

Properties (v) and (vi) of the candidate compounds (substances) are essentially reflected by the experiments of FIG. 6 with the corresponding methods in section “Material and Methods”, chapter “Immunoprecipitation and GST-pulldown” as they are described.

The nucleotide status of Rab5 as mentioned in (iv) above defines to which of GDP or GTP the Rab5 protein is bound.

Thus, the inventors provide an assay (in vivo) to screen for anti-proliferative drugs, the assay comprising the steps of:
(a) contacting cells of a primary cell culture or of an established cell line with a candidate substance,
(b) subsequently or concomitantly with a candidate substance, contacting the cells with a growth factor,
(c) processing the cells according to standard procedures for immunofluorescence staining to detect APPL1 and APPL2 using an anti-APPL1 and/or 2 antibody, or alternatively using GFP-tagged APPL proteins stably or transiently expressed by the cells via transfection,
(d) assessing the degree of colocalisation of APPL1 and/or 2 and the growth factor, the solubilisation of APPL1 and/or 2 (intended as translocation from the hermesome membrane to the cytosol) and their translocation to the nucleus,
(e) repeating steps (b) to (d) with cells not previously treated with the candidate substance, and
(f) comparing the degree of. colocalisation of APPL1 and/or 2 and the growth factor, the solubilisation of APPL1 and/or 2 and their translocation to the nucleus between the cells not previously treated with the candidate substance (untreated cells) and cells treated with the candidate substance (treated cells),
   wherein an altered degree of colocalisation of APPL1 and/or 2 and the growth factor, i.e: reflecting the sorting and transport of the growth factor and its receptor into hermesomes, an altered solubilisation of APPL1 and/or 2 and/or their altered translocation to the nucleus in the treated vs. the untreated cells identifies the candidate substance as an anti-proliferative drug.

According to a specific embodiment of the in vivo-assay, an decreased degree of colocalisation of APPL1 and/or 2 and the growth factor, an decreased solubilisation of the APPL proteins and/or their decreased translocation to the nucleus in the treated vs. the untreated cells identifies the candidate substance as an anti-proliferative drug.

According to another specific embodiment, the in vivo-assay is performed with epidermal growth factors (EGFs) and neuregulin (NRG) family, with fibroblast growth factors (FGFs), with transforming growth factors-β (TGFs-β) and the family, with transforming growth factor-α (TGF-α), with insulin-hke growth factor-I (IGF-I) and -II (IGF-II), with tumour necrosis factor-α (TNF-α) and -β (TNF-β), with vascular endothelial growth factor (VEGF), nerve growth factor (NGF), with hepatocyte growth factor/scatter factor, pleiotrophin, oncostatin M (OSM), with angiogenic factors (angiogenins), ephrins, interleukins (ILs) 1-13, interferons (INFs) α, β, γ, with colony stimulating factors (CSFs), with erythropoietin (EPO), with platelet-derived growth factor (PDGF) and/or with any other growth factors that may signal via the hermesome.

According to another embodiment of the assay the growth factor and/or the antibody/antibodies are/is labelled, preferably fluorescently, and/or step (d) of assessing is performed by fluorescence microscopy.

In this assay, hermesomes play their usual role as they do in a living cell within an organism. The hermesomes are accessible to the growth factor and possibly to the candidate substance via endocytosis or, alternatively, the substance can penetrate into the cell cytosol and contact the cytoplasmic surface of the plasma membrane from where transport vesicles directed to hermesomes originate and/or of the hermesome itself. In other words, the assay involves an in vivo-use of hermesomes for the screening for anti-proliferative drugs.

Another aspect of the present invention is an anti-proliferative drug, identified and/or isolated according to the assay to screen for anti-proliferative drugs, as described above.

Still another aspect of the present invention is the use of such anti-proliferative drug in the manufacture of a pharmaceutical to treat cancer/tumour diseases. According to a particular embodiment, treatment occurs by an inhibition of proliferation and/or induction of apoptosis in cancer/tumour cells.

In addition, the inventors have also developed a method to isolate hermesomes. In view of that fact, another aspect of the invention relates to an in vitro-assay to screen for such anti-proliferative drugs. In particular, the present invention relates to an in vitro-assay to screen for anti-proliferative drugs, the assay comprising the steps of:
(a) isolating hermosomes from cells of a cell culture, in particular by density gradient centrifugation,
(b) restoring their functionality by contacting the hermesomes with cytosol, an ATP-regenerating system and either or both of GTP and GDP,
(c) modulating their function in cell proliferation and/or apoptosis by substances that modulate
   1) the recruitment of Rab5 on hermesome,
   2) the activity of Rab5 (intended as fraction of the molecule in the GTP-bound form and GTP hydrolysis activity) and, consequently, the release of APPL1 and/or APPL2 from hermesomes, and
   3) the ability of the released APPL proteins to interact with the NuRD/MeCP1 complex or its associated factors such as p53.

This is determined by:
1) assaying the capabilities of hermesomes to recruit endogenous as well as exogenous Rab5 by contacting them with the recombinant Rab5-GDI complex, that allows the delivery of Rab5 to the membrane, followed by re-isolation of hermesomes by centrifugation and analysing the levels of Rab5 by Western blot;
2) analysing the levels of APPL1 and/or APPL2 on the hermesomes by Western blotting and thereby assaying the levels of Rab5 activation (the amount of APPL1 and/or APPL2 on hermesomes is proportional to the amount of Rab5 bound to GTP); and
3) quantifying the association of APPL1 and/or APPL2 with the aforementioned as well as other proteins by immunoprecipitation and GST-pull down as described in the methods.

This assay will be performed comparing hermesomes isolated from cells previously treated with or without the growth factor (stimulated or non-stimulated cells), with or without a candidate substance (treated or untreated cells) or exposed to a candidate substance after isolation.

The present invention will be explained to some more detail by reference to FIGS. 1 to 10, which are briefly discussed below.

FIG. 1: APPL1 and APPL2 are Rab5 effectors. a, The pattern of cytosolic proteins interacting specifically with Rab5-GTPγS. GST-Rab5 affinity chromatography was performed as described ¹⁹ and PAGE-separated proteins stained by Coomasie. b, Domain structure of APPL1 and APPL2 proteins. c, APPL1 and APPL2 interact specifically with Rab5-GTPγS. In vitro translated, [³⁵S]methionine labelled APPL proteins were incubated with glutathione-sepharose beads loaded with GST-Rab proteins in the GDP or GTPγS forms, as described²¹. Bound proteins were analysed by SDS-PAGE and autoradiography. d, Anti-APPL1 and anti-APPL2 peptide antibodies recognise single bands in HeLa cytosol by Western blot. e,. Endogenous APPL1 localises to Rab5Q79L-enlarged endosomes in vivo. HeLa cells were transfected with Rab5Q79L and stained with antibodies against APPL1. The transfected cell is indicated with an asterisk. f-g, Distribution of endogenous APPL1 and APPL2 in HeLa cells, stained with specific antibodies as indicated. Individual confocal sections are shown in e-g. Scale bar 20 μm.

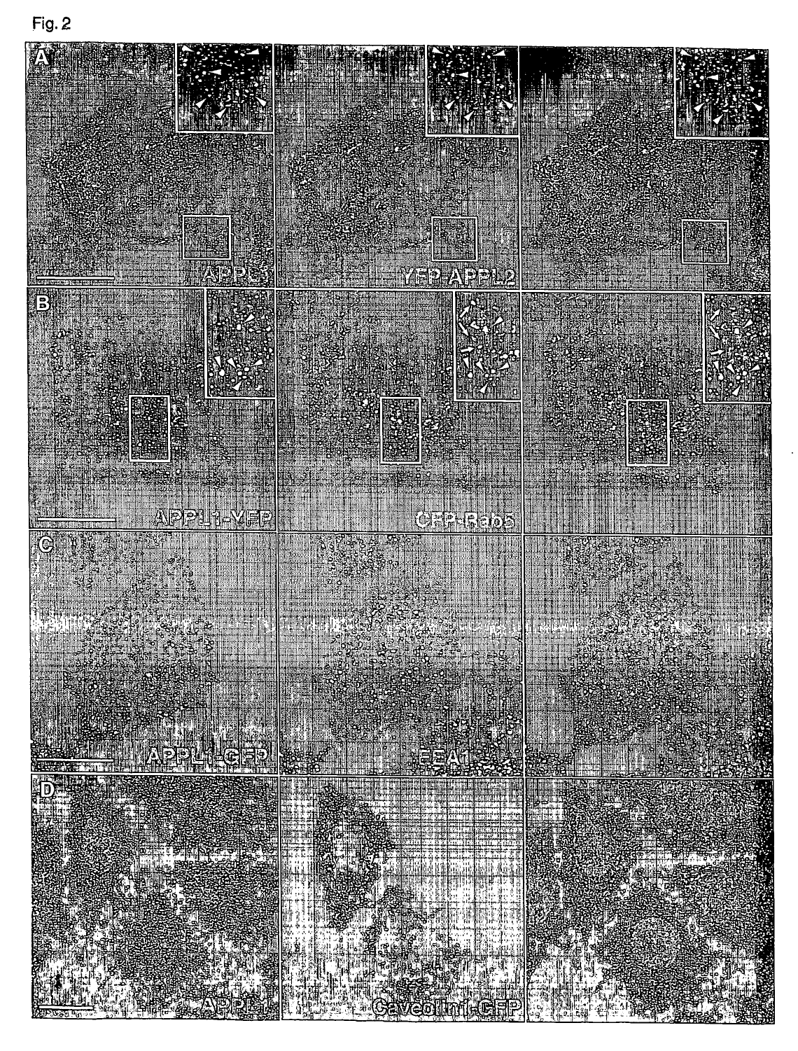

FIG. 2: Morphological characterisation of intracellular structures labelled by APPL1 and APPL2. APPL1 and APPL2 colocalise with each other (a) and Rab5 (b) but not EEA1 (c) or caveolin (d) in peripheral punctuate structures. HeLa cells were transfected with the C/G/YFP constructs, fixed and stained with anti-APPL1 or anti-EEA1 antibodies, as indicated. Arrowheads in panels a and b indicate the structures shared between APPL1 and YFP-APPL2 or Rab5, respectively. Arrows in panel b mark the Rab5-positive structures, which do not contain APPL1. Individual confocal sections are shown in all panels. Scale bar 20 μm.

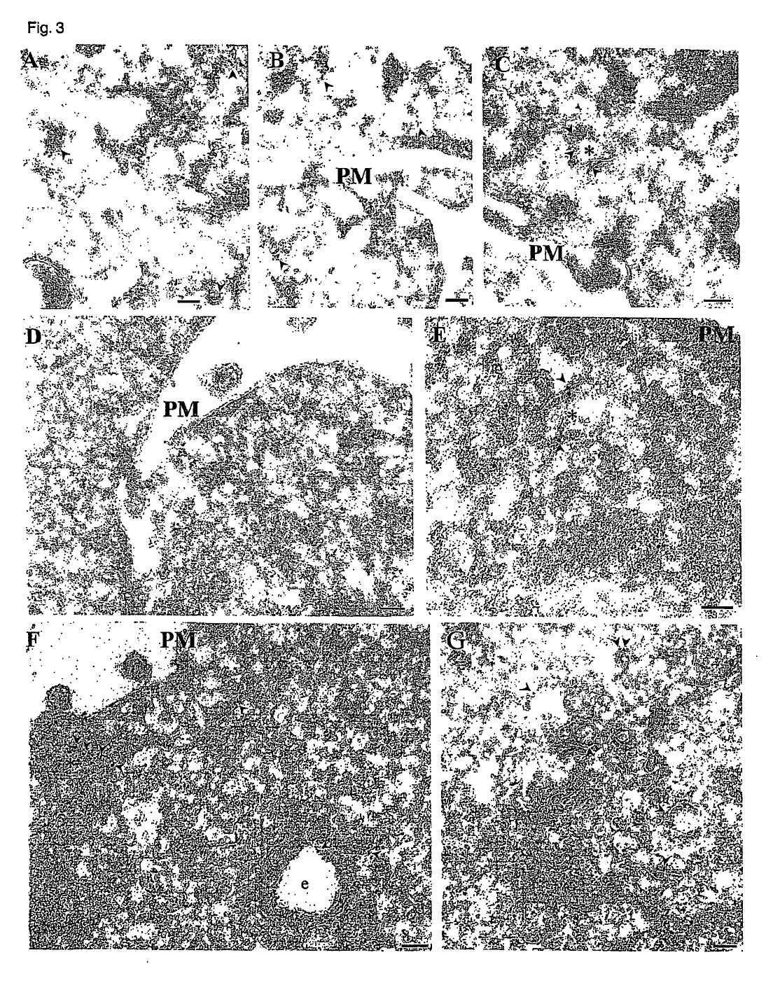

FIG. 3: Electron microscopic localisation of endogenous and expressed epitope-tagged APPL1. a-c, Serum-starved A431 cells were fixed with paraformaldehyde and processed for frozen sectioning. Sections were labelled with antibodies to APPL1 followed by 10 nm protein A-gold. Specific labelling (arrowheads) is associated with structures with variable morphology close to the plasma membrane (PM). The labelling is associated with membrane-bound structures (particularly evident in the structures labelled with asterisks—also see panels e and f). d-g, BHK cells were transfected with APPL1-GFP and processed for frozen sectioning. Sections were labelled with antibodies to GFP followed by 10nm protein A-gold. The highest expressing cells showed strong labelling throughout the cytoplasm (d, cell on the right). Panels e-g show representative sections from cells expressing low but significant levels of APPL1-GFP. Labelling is concentrated below the plasma membrane in small membranous structures with variable morphology (arrowheads). Some labelling of similar structures in the perinuclear area of the cell was also observed (panel g). Classical early endosomes, recognised by their characteristic ring-shape and multivesicular domains generally showed poor labelling for APPL1-GFP in the low expressing cells (e.g. see panel f, endosome labelled ‘e’). Scale bar 100 nm.

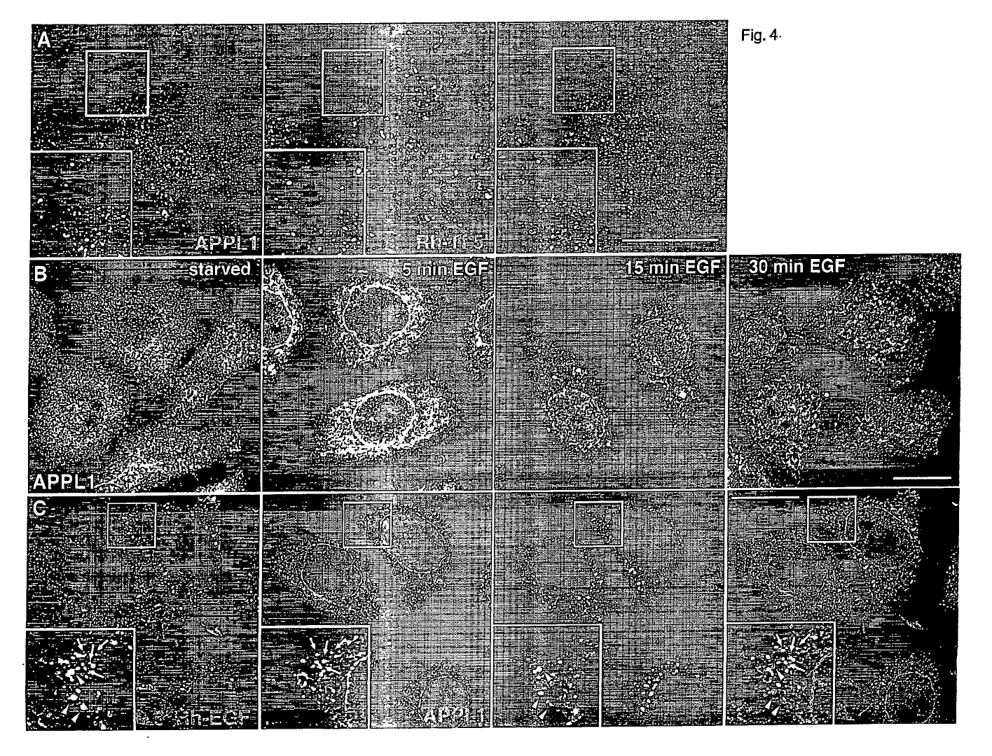

FIG. 4: EGF, but no transferrin, is internalised into APPL structures and causes APPL1 redistribution. a, HeLa cells were serum-starved for 1 h and incubated with 30 μg/ml of rhodamine-transferrin (Rh-Tf) for 5 min at 37° C., fixed and stained with anti-APPL1 antibodies. The degree of colocalisation between APPL1 and Rh-Tf was not increased upon longer intemalisation times (30 min). b, HeLa cells were serum-starved overnight and incubated with 1 μg/ml Rh-EGF for 5, 15 or 30 min at 37° C., fixed and stained with anti-APPL1 antibodies. c, HeLa cells were treated with Rh-EGF for 5 min, fixed and stained with anti-APPL1 or anti-EEA1 antibodies. Arrowheads in panel c indicate the structures labelled by EGF and EEA1, while arrows indicate the EGF- and APPL1-positive vesicles. Individual confocal sections are shown. Scale bar 20 μm.

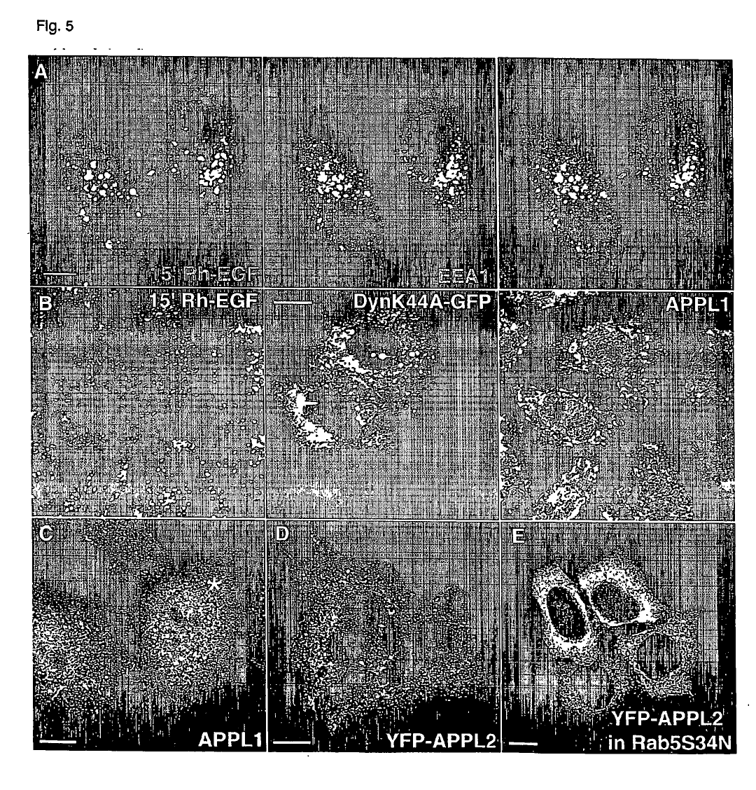

FIG. 5: Release of APPL1 from membranes is dependent on Rab5-GTP but not Dynamin. a-b, Control HeLa cells (a) or cells transfected with Dynamin^{K44A}-GFP (b) were serum-starved overnight, incubated with Rh-EGF for 15 min at 37° C, fixed and stained with anti-EEA1 or -APPL1 antibodies, as indicated. c-e, APPL proteins associate with the novel compartment in a Rab5-dependent manner. c, HeLa cells with transfected with Rab5S34N, fixed and stained with anti-APPL1 antibodies. The transfected cell is indicated with an asterisk. d-e, HeLa cells were transfected with YFP-APPL2 alone (d) or in combination with Rab5S34N (e). Only the localisation of over-expressed YFP-APPL2 is shown. Scale bar 10 μm.

FIG. 6: APPL proteins interact with the components of the nucleosome remodelling and histone deacetylase complex NuRD/MeCP1. a, Coomassie-stained proteins co-immunoprecipitated from detergent extracts of HeLa membrane fraction by anti-APPL1 antibody. b, Western blot detection of PID/MTA2 and RbAp46 immunoprecipitated from HeLa nuclear extracts by antibodies against APPL1 and APPL2 and a preimmune (PI) serum. c, GST pulldown of proteins interacting with APPL1 and APPL2. HeLa nuclear extracts were incubated with the beads containing GST alone or fused to APPL1 or APPL2. PID/MTA2 and RbAp46 retained on the columns were detected by Western blot.

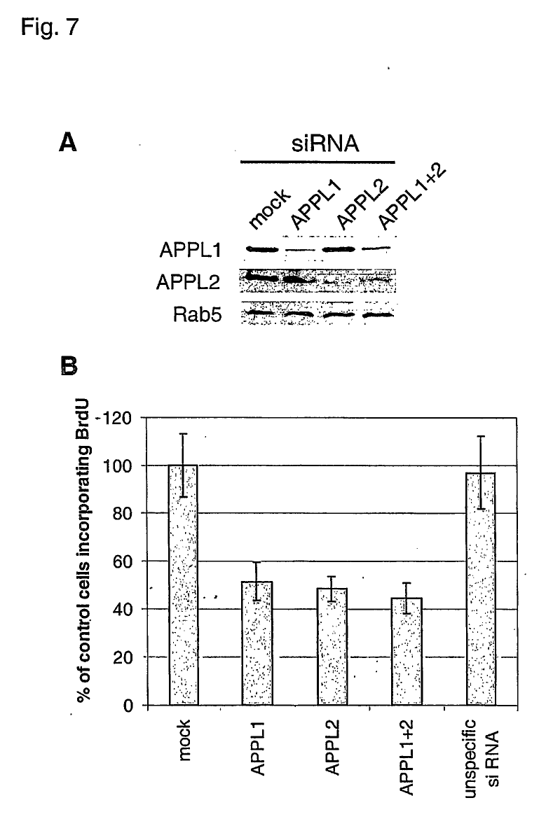

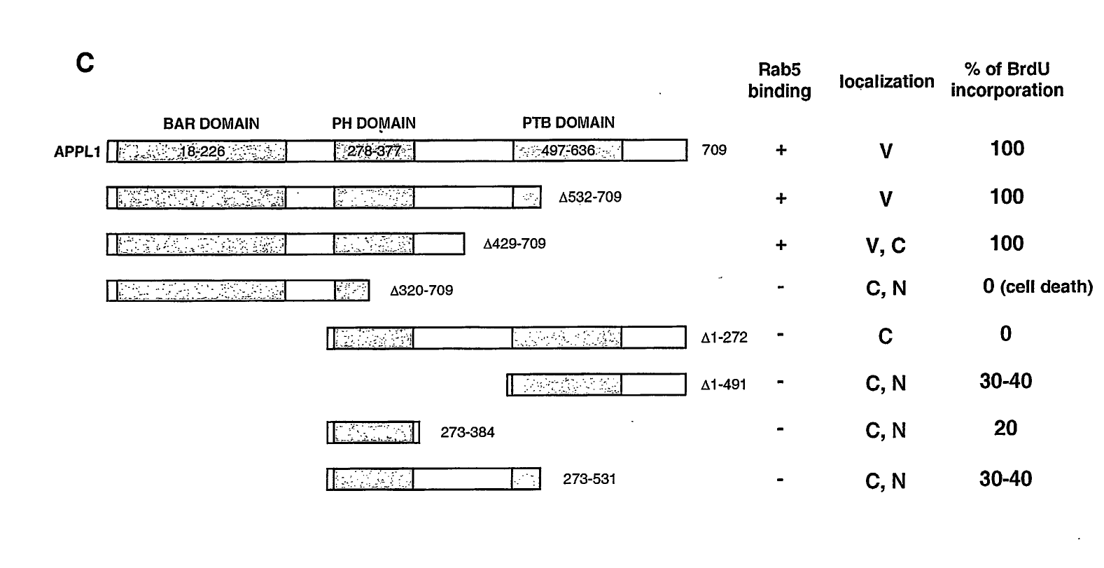

FIG. 7: APPL1 and APPL2 are required for cell proliferation and Rab5 binding is essential for their function. a, Reduced levels of APPL1 and APPL2 48 hours after transfecting HeLa cells with siRNA oligos, as detected by Western blot. b, Histogram showing the percentage of cells incorporating BrdU (1 h pulse) 48 h after transfection with siRNA oligos. Typically, about 50-60% of control cells showed BrdU incorporation under these conditions. c, Schematic representation of APPL1 deletion mutants. Rab5 binding was assessed biochemically as in FIG. 1c. All mutants were stably expressed in reticulocyte lysates. Intracellular localisation was tested by transfecting YFP-fusion constructs in HeLa cells (N, nuclear; C, cytosolic; V, vesicular). Percentage of BrdU incorporation was determined in cells transfected with YFP-fiision constructs (BrdU incorporation in cells transfected with YFP alone was set to 100%).

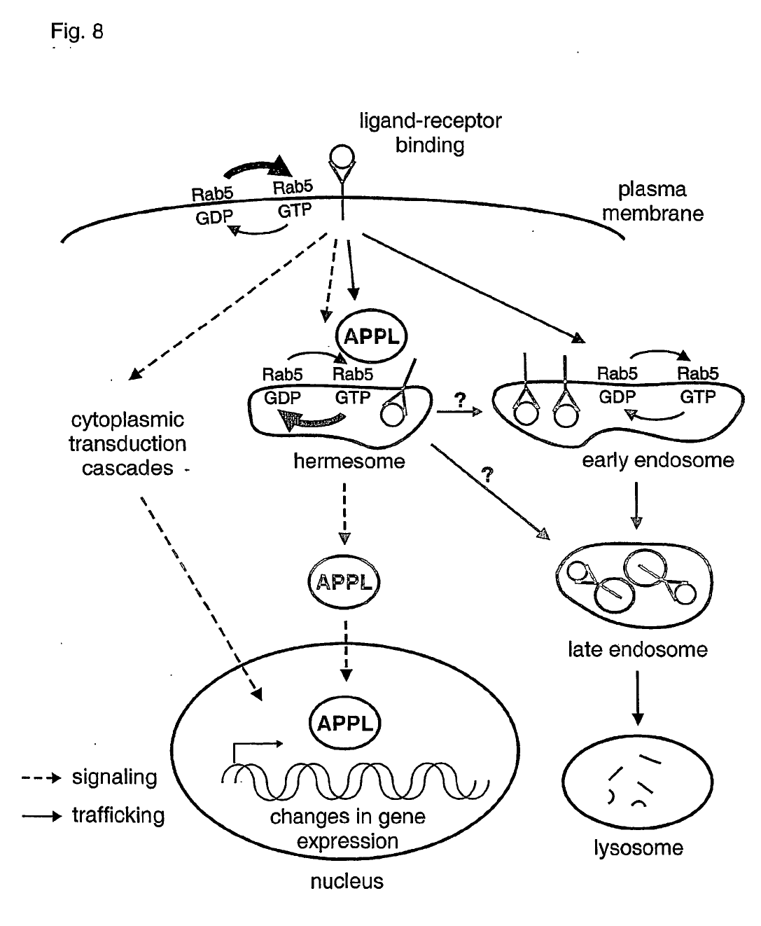

FIG. 8: Model of the integration of the novel organelle into intracellular signalling pathways. A spatial separation of Rab5 between different organelle pools provides a possibility of an independent regulation of its GTPase cycle in various locations.

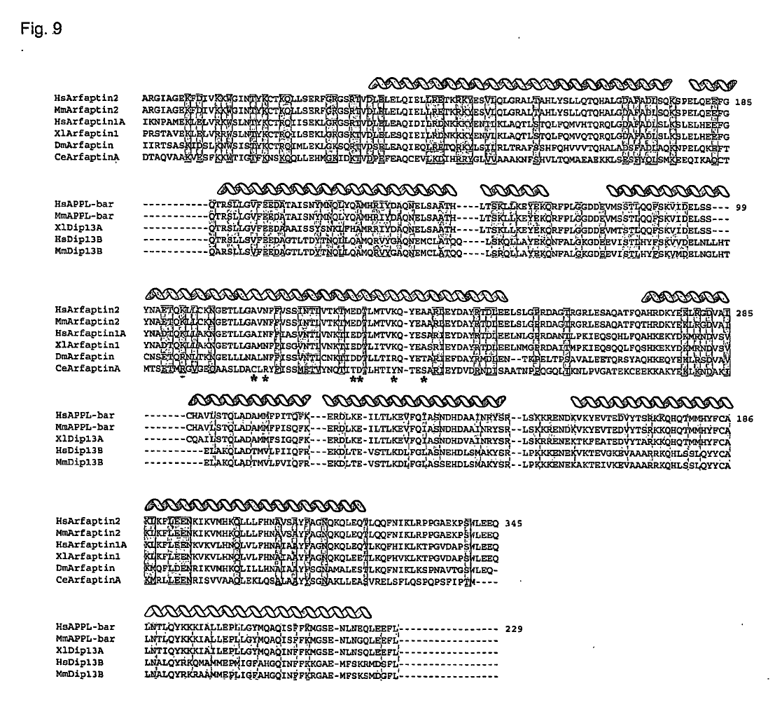

FIG. 9: Identification of a BAR domain in APPL proteins. Multiple sequence alignment of the families of APPLs and Arfaptins. Conservation between both families is indicated by yellow, within the APPL- and Arfaptin- subfamilies in green and blue, respectively. Secondary structural elements are indicated at the top of the alignment. Asterisks indicate sites of interaction between Arfaptin2 and Rac-GDB, as determined by the crystal structure (pdb code 1I4L). Numbering according to HsArfaptin2 and HsAPPL-BAR, respectively. Dip13B=APPL2. Accession numbers: HsArfaptin2: NP_{—}036534.1; MmArfaptin2: NP_{—}084078.1; HsArfaptin1A: NP_{—}055262.1; X1 Arfaptin1: AH45010.1; DmArfaptin: NP_{—}650058.1; CeArfaptinA: S40749; HsAPPL: NP_{—}036228.1; MmAPPL: NP_{—}660256.1; X1 Dip13A: AAH46747.1; HsDip13B: NP_{—}060641.2; MmDip13B: NP_{—}660255.1

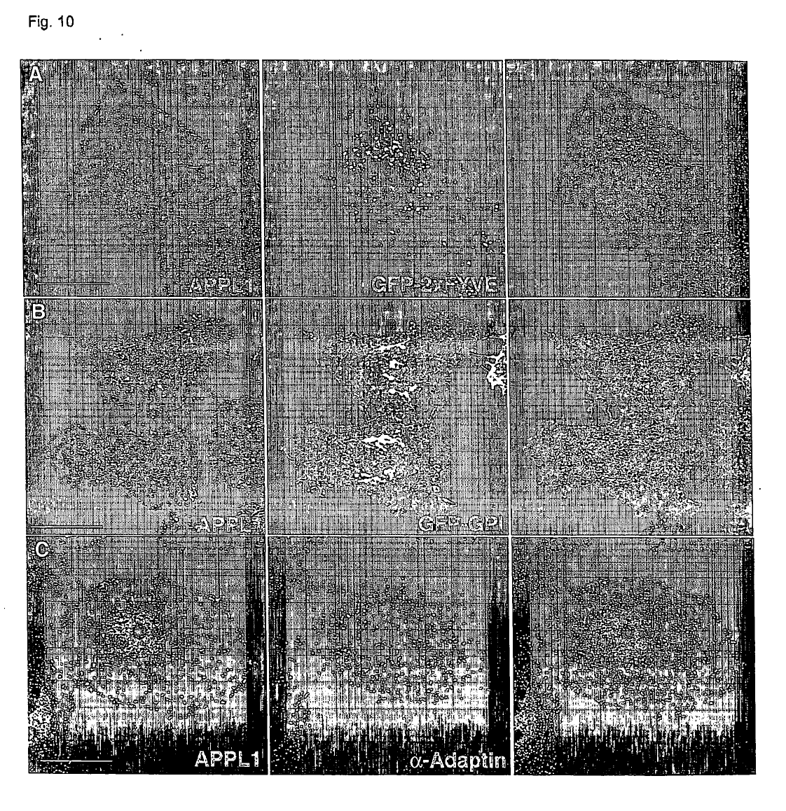

FIG. 10: Intracellular structures labelled by APPL1 and APPL2 do not contain endocytic markers. a-b, HeLa cells were transfected with GFP-constructs as indicated and stained with antibodies against APPL1. c, Distribution of endogenous APPL1 and α- adaptin, detected by specific antibodies. Individual confocal sections are shown in all panels. Scale bar 20 μm.

### IDENTIFICATION OF TWO NOVEL Rab5 EFFECTORS

In a search for new Rab5 effectors, nanoelectrospray tandem mass spectrometry revealed that one of the most abundant proteins (˜80 kDa) affinity purified on a GST-Rab5:GTPγS column (FIG. 1A)19 corresponded to APPL1. Further sequencing of the GST-Rab5:GTPγS eluate from HeLa cytosol revealed a protein of 664 amino acids and 54% identity to APPL1 (recently named DIP13β, accession no. NM_{—}018171). According to the original nomenclature, the latter protein is referred to here as APPL2. Both APPL proteins are encoded by two different genes (on human chromosomes 3 and 12, respectively) but share the same domain organisation, with a central pleckstrin homology (PH) domain and a phosphotyrosine binding domain (PTB) at the C-terminus, involved in binding AKT and DCC (FIG. 1B). A potential nuclear localisation signal on APPL2 (¹⁵¹PKKKENE¹⁵⁷) was detected by PSORT II program³². Furthermore, by SMART-analysis³³ the inventors identified the presence of a BAR domain (BIN1/Amphiphysin/RVS167;³⁴ in the N-terminal part of APPL1 (FIG. 9). Given the relatively high homology between APPL1 and APPL2 in this region (54% identity and 74% similarity), it can be assumed that APPL2 also contains a BAR-domain. Interestingly, PSI-Blast searches 35 with the BAR-domain of APPL1 or APPL2, as well as structural predictions using 3D-PSSM ³⁶ indicate that the BAR-domain is distantly related to Arfaptins, which bind ARF and Rac GTPases^{37,38} (FIG. 9).

To test whether the interaction with Rab5 is direct and specific, the inventors cloned and in vitro translated both APPL proteins to measure their ability to bind various recombinant GST-tagged Rab proteins. As shown in FIG. 1C, both APPL1 and APPL2 strongly bound Rab5-GTPγS but neither Rab5-GDP nor any other endocytic Rab proteins tested (Rab4, Rab7 or Rab11), indicating that they are specific effectors of Rab5. In an attempt to confirm that APPL1 and APPL2 colocalise with Rab5-GTP in vivo, the inventors expressed in HeLa cells the constitutively active Rab5Q79L mutant that induces the formation of expanded endosomes³⁹. They raised antibodies against the C-terminal peptides of both proteins which recognise endogenous levels of the corresponding antigens and do not exhibit any cross-reactivity between the two proteins (see below). Both endogenous APPL (FIG. 1E) and APPL2 accumulated on the enlarged endosomes. Thus, APPL proteins specifically interact with Rab5-GTP in vitro and localise to membranes harbouring this protein in vivo.

APPL1 and APPL2 Localise to a Novel Cytoplasmic Organelle

In contrast to other Rab5 effectors exhibiting a typical endosomal staining pattern, it was surprising to observe a more complex intracellular distribution of APPL1 and APPL2. In HeLa (FIG. 1F), A431 and BHK cells, APPL1 localises to punctate structures dispersed in the cytoplasm but mostly concentrated underneath the plasma membrane. Similar structures are also labelled for APPL2 (FIG. 1G). In addition, both proteins are present in the nucleus. Whereas APPL2 is particularly enriched in the nucleus with respect to the cytoplasmic structures, the intensity of the nuclear staining of APPL1 varies between individual cells. It has also been noted by the inventors that anti-APPL1 but not APPL2 antibodies occasionally label mitochondria and this staining correlates with the progressive increase of cell passages in culture. The significance of this staining remains at present unclear.

Given the complexity of the staining pattern it was essential to exclude antibody artefacts. Four lines of evidence validate the specificity of the staining. First, both anti-APPL1 and -APPL2 antibodies recognise single bands corresponding to the predicted protein size in HeLa cytosol by Western blot (FIG. 1D). Second, the immunofluorescence staining was abolished upon preincubation of the antibodies with the respective peptide. Third, knocking down both genes by siRNA drastically reduced or abolished both immunofluorescence and Western blot signals (FIG. 7A). Fourth, APPL1 and APPL2 tagged with GFP at the C-terminus colocalised with the endogenous proteins in the peripheral structures and on Rab5Q79L enlarged endosomes (FIG. 2A). Since the tagged proteins were not targeted to the nucleus (FIG. 2A-B), as previously documented for APPL1³⁰, the inventors limited the use of these constructs to label the cytoplasmic structures.

Having established the authenticity of the staining pattern, the inventors analysed the APPL-positive peripheral structures in more detail. Endogenous APPL1 largely colocalises with YFP-APPL2 in the same punctate structures (FIG. 2A), which are also positive for CFP-Rab5 (FIG. 2B). Surprisingly, no colocalisation between APPL1 and markers of early endosomes (EEA1, FIG. 2C) was observed. Importantly, the APPL structures underlying the plasma membrane were clearly negative and separated from EEA1-positive endosomes by distances in the micrometer range, excluding the possibility that APPL could mark a subdomain of early endosomes, as reported for Rab5, Rab4 and Rab11⁴⁰. Consistently, immunodepletion of both APPL1 and APPL2 from HeLa cytosol did not inhibit heterotypic and homotypic early endosome fusion⁴¹.

Does the APPL compartment represent any other established organelle of the biosynthetic or endocytic pathway? Further morphological analysis eliminated this possibility. APPL1 is not present in Rab11-positive early and recycling endosomes. Furthermore, the distribution of APPL1-positive structures is unaffected by treatment with wortmannin or brefeldin A, which selectively affect the morphology of Rab5- and Rab4/Rab11-positive endosomes, respectively^{40,42}. It has been further established that APPL structures are neither enriched in the endosomal phosphoinositide PI(3)P, nor in PI(4,5)P₂, PI(3,4,5)P₃ or in PI(4)P, as revealed by the specific lipid probes (2×FYVE domain, PH domains of PLCδ, AKT1 or FAPP1, respectively) (FIG. 10).

The inventors confirmed lack of any colocalisation with various ER and Golgi markers (Sec61-GFP, β-COP, TGN38 or γ-adaptin). Despite a possible resemblance of peripheral APPL structures with caveolae and caveosomes, no colocalisation with Caveolin1-GFP⁴³ was found (FIG. 2D). The APPL1-labelled structures were negative for GFP-glycosylphosphatidylinositol (GPI), a marker of distinct tubular-vesicular endosomes⁴⁴ (FIG. 10), α-Adaptin (FIG. 10) or Clathrin, markers of endocytic Clathrin-coated vesicles, and the late endosomal Rab7.

The inventors next examined the distribution of APPL1 by immunoelectron microscopy on frozen sections. As shown in FIG. 3A-C, specific labelling was associated with membrane-bound structures close to the plasma membrane, which did not show the typical morphology of classical early endosomes. Upon over-expression of APPL1-GFP, high labelling throughout the cytoplasm was observed in the highest expressing cells (FIG. 3D). In lower expressing cells, labelling was predominantly associated with membranous structures close to the plasma membrane (FIG. 3F) and in the perinuclear area (FIG. 3G). Structures with the typical morphology of early endosomes showed very low or undetectable labelling (FIG. 3F). These data clearly establish that APPL structures are membrane bound, consistent with the fact that they contain a membrane marker such as Rab5 (FIG. 2B) and that APPL1 was detectable in membrane preparations isolated by floatation on density gradient. Cumulatively, the morphological studies indicate that the APPL proteins are localised to a novel Rab5-positive membrane-bound organelle.

EGF is Internalised into APPL Structures and Causes APPL1 Redistribution

As a next step, the inventors set out to determine whether the APPL structures are accessible to endocytic cargo intemalised for different periods of time either via receptor-mediated (transferrin) or by fluid-phase endocytosis (dextran). Only a very low degree of APPL1 colocalisation with intemalised transferrin (FIG. 4A) and no significant labelling with endocytosed dextran at any time point were observed, arguing that APPL-positive structures are not pinosomes. Given that the machineries responsible for constitutive (transferrin) and ligand-induced (growth factors) endocytosis can be differentially regulated 6, the inventors tested whether rhodarnine-labelled EGF (Rh-EGF) could access APPL structures. Cells were serum-starved overnight and Rh-EGF was intemalised for 5, 15 or 30 min. in order to progressively label Clathrin-coated vesicles, early endosomes, late endosomes/lysosomes. Unexpectedly, the inventors observed that the APPL1 distribution changed dramatically upon serum starvation and EGF stimulation (FIG. 4B). In serum-starved cells, APPL1 was restricted to the punctate structures in cytosol and absent from the nucleus. In sharp contrast, upon treatment of cells with Rh-EGF for 5 min, APPL1 partly translocated from the peripheral structures to the cytoplasm, became particularly enriched on the nuclear envelope and began to appear in the nucleus. Within 15 min of Rh-EGF treatment APPL1 shifted from the cytoplasmic structures to the nucleus and after 30 min its accumulation in the nucleus subsided and the typical APPL1-positive puncta underlying plasma membrane reappeared. The response of APPL1 to EGF indeed correlates with the accessibility of APPL1-positive membranes to this growth factor. FIG. 4C shows that after 5 min of internalisation a fraction of Rh-EGF was present in fine puncta harbouring APPL1, in addition to EEA1-positive early endosomes and, presumably, EEA1-negative Clathrin-coated vesicles¹⁸. The extent of colocalisation varied depending on the degree of APPL1 mobilisation from the peripheral vesicles. At 15 min, Rh-EGF appeared in EEA1-containing early endosomes (FIG. 5A) that expanded in size as shown previously¹² and colocalisation with APPL1 was no longer detectable. These data illustrate two main points of the present invention. First, the APPL structures are specialised endosomes as they are selectively accessible to endocytic cargo such as EGF, although they do not constitute its major intemalisation route. Second, APPL1 undergoes regulated cycles of redistribution between cytoplasmic vesicles and the nucleus in response to EGF. Unfortunately, the exclusion of GFP-labelled APPL from the nucleus prevented the possibility to capture this interesting process by video microscopy.

Subsequently, the inventors tested whether the APPL1 cycle in response to EGF internalisation depended on Dynamin. They over-expressed a dominant negative mutant of Dynamin II (K44A) and assayed Rh-EGF uptake in cells serum-starved overnight. Strikingly, although Dynamin^{K44A} blocks the transport of EGF into early and late endosomes, as evidenced by the lack of enlarged endosomes labelled with Rh-EGF (FIG. 5, compare panel A with B, 15 min Rh-EGF), a fine punctate labelling of EGF resembling the APPL1 staining and underlying plasma membrane was observed (FIG. 5B). Importantly, Dynamin^{K44A} does not affect the translocation of APPL1 to the nucleus (FIG. 5B). On the contrary, APPL1 is more readily released from the membranes in Dynamin^{K44A} expressing cells compared with control cells. These results demonstrate that EGF intemalisation into APPL-positive endosomal structures operates Dynamin-independently and that EGF-dependent release of APPL from these structures can occur upon impairment of Dynamin function.

GTP Hydrolysis by Rab5 Releases APPL1 from Endocytic Structures in Response to Extracellular Stimuli

The next question the present inventors posed was by which mechanism APPL1 might be released from its cytoplasmic vesicles. GTP hydrolysis on Rab5 could potentially disengage APPL1 from the membranes since APPL binding to Rab5 is GTP-dependent. To test this possibility the inventors performed three experimental approaches. First, they examined the effect of over-expression of Rab5S34N, a mutant preferentially stabilised in the GDP conformation, on the localisation of endogenous APPL1. A dramatic redistribution of endogenous APPL1 (FIG. 5C) or expressed YFP-APPL2 (FIG. 5D-E) from the punctate structures to the cytosol was indeed observed in Rab5S34N expressing cells. Second, over-expression of the Rab5 GAP RN-tre¹⁴ caused a substantial displacement of APPL1 from the peripheral structures, consistent with the reduction of the pool of active Rab5 in these cells. Third, they took advantage of the fact that p38MAPK activation by oxidative stress results in phosphorylation of RabGDI, thereby causing extraction of Rab5 from membranes, accumulation of Rab5:GDP-GDI complex in cytosol, and specific loss of effectors, such as EEA1, from the early endosomes⁴⁵. Consistently, upon treatment of HeLa cells with H₂0₂ for 15 minutes a progressive loss of APPL1 from the peripheral structures and its accumulation in the nucleus could be observed. These results provide independent evidence that active Rab5 is a primary determinant of APPL1membrane localisation, and GTP hydrolysis or reduction in Rab5-GTP levels on the membrane release APPL1 into cytosol. Moreover, they establish that oxidative stress, similarly to EGF stimulation, is another signalling pathway that relocates APPL1 to the nucleus.

APPL Proteins Interact with Components of Nucleosome Remodelling and Histone Deacetylase Complex NuRD/MeCP1 and are Required for Cell Proliferation

To gain further insights into the function of APPL1 the inventors undertook a search for interacting partners by co-immunoprecipitation experiments from cytosol and detergent extracts of HeLa cells. Whereas no proteins were co-immunoprecipitated with APPL1 from cytosol, a number of proteins were recovered from the detergent extract (FIG. 6A). Surprisingly, mass spectrometry sequencing revealed the presence of PID/MTA2, p66, HDAC1 and/or HDAC2 (identified through common peptides), RbAp46, RbAp48 and MBD3, namely 6 out of 10 components of the nucleosome remodelling and histone deacetylase NuRD/MeCP1 complex⁴⁶. PID/MTA2 (p53 target protein in the deacetylase complexes/metastasis associated protein 2;⁴⁷) was one of the most abundant proteins in the immunoprecipitate. Given the reported nuclear localisation of the interacting proteins, the inventors confirmed the specificity of the co-immunoprecipitation this time using HeLa nuclear extracts. Western blot analysis (FIG. 6B) showed that anti-APPL1 antibodies but not pre-immune serum strongly and specifically co-immunoprecipitated PID/MTA2 protein and, to a lesser extent, also RbAp46. A similar interaction using APPL2 antibodies could not be observed, presumably due to their low efficiency in immunoprecipitation. The inventors furthermore confirmed these interactions by GST pull-down experiments applying nuclear extracts to columns with immobilised GST alone or fused to APPL proteins (FIG. 6C). PID/MTA2 and RbAp46 were specifically bound to GST-APPL1 but not GST alone. Interestingly, the inventors also recovered these proteins on the GST-APPL2 column, suggesting that both APPL proteins can interact with the components of NuRD/MeCP1 in the nucleus.

It has been known for some time that histone deacetylase activities are required for cell cycle progression and development⁴⁸⁻⁵⁰. The identification of the NuRD/MeCP1 complex as binding partner together with the nuclear localisation of APPL1 and APPL2 prompted the present inventors to investigate their function with respect to cell proliferation. They assayed DNA synthesis under downregulation of endogenous APPL proteins by RNA interference⁵¹. Forty-eight hours after transfecting the cells with small interfering RNA oligonucleotides specific for APPL1 or APPL2, a pronounced reduction in protein levels of APPL1 and/or APPL2 could be observed, as evidenced by Western blot (FIG. 7A) and immunofluorescence analysis. Strikingly, by measuring BrdU incorporation it was further observed that knock-down of either APPL1 or APPL2 resulted in a 50% reduction in the number of cells entering S-phase in comparison with control cells (mock treated or transfected with unrelated siRNA; FIG. 7B). The inhibitory effects on DNA synthesis elicited by knock-down of either APPL1 or APPL2 were not additive (FIG. 7B). These data argue that the two proteins cannot substitute for each other. Importantly, no increase in cell death was evident under these conditions, as determined by Tryptan blue staining. Collectively, the interaction with the NuRD/MeCP1 complex together with the effects on DNA replication are convincing evidence for both APPL proteins exhibiting essential functions in a pathway required for cell proliferation.

Binding to Rab5 is Indispensable for the Functional Cycle of APPL1

In order to provide a solution to the object posed, the inventors further wished to assay the role of Rab5 in the regulation of cell proliferation by APPL proteins. Although over-expression of Rab5S34N has been previously shown to inhibit proliferation of endothelial cells and keratinocytes²⁹, the profound pleiotropic effects of Rab5 mutants on endocytosis and cellular homeostasis make such results difficult to interpret. Thus, the inventors resolved instead to test whether Rab5 binding is important for APPL function in the regulation of cell proliferation.

They first conducted deletion mutagenesis and in vitro binding studies to identify sequences engaged in Rab5 binding on the APPL1 molecule (FIG. 7C). Based on the homology of the BAR domain to Arfaptins they focused on this region of APPL1 as the potential binding site. Strikingly, the presence of both BAR and PH domains (residues 1-428) was found to be necessary for binding to Rab5-GTP, suggesting that one domain may stabilise the other or both may co-operatively bind Rab5. In contrast to some PH domains exhibiting high affinity and specificity for certain phosphoinositides⁵², the PH domain of APPL1 is not targeted to any cellular membranes remaining cytosolic. Remarkably, when over-expressed in vivo as fluorescently tagged proteins, only mutants capable of interacting with Rab5 exhibit membrane localisation, further underscoring the function of Rab5 as a primary determinant of APPL localisation to the cytoplasmic structures (FIG. 7C).

The inventors further investigated the effect of the truncation mutants on DNA synthesis, as measured by BrdU incorporation (FIG. 7C). While the over-expressed wild-type protein or truncation mutants capable of Rab5 binding (Δ532-709, A429-709) did not affect the rate of DNA synthesis, all mutants unable to interact with Rab5 elicited some inhibitory effects on this process. In particular, the overproduction of the APPL1A1-272 mutant protein lacking the BAR domain and unable to bind Rab5 completely blocked BrdU incorporation in transfected cells. Since the mutant protein accumulated in the cytosol and was excluded from the nucleus, its anti-proliferative activity in all likelihood depends on the sequestration of yet unidentified soluble factors acting prior to nuclear import of APPL1. Collectively, these data demonstrate that 1) binding to Rab5 and 2) cytoplasmic interactions induced by translocation from the vesicles to the nucleus constitute essential steps of the functional cycle of the APPL1 protein. Moreover, over-expression of the APPL1Δ320-705 mutant comprising the BAR domain caused increased cell death, indicating that interference with the activity of APPL may induce a pro-apoptotic effect.

In this application, discovery of a novel cell organelle involved in a new signal transduction pathway between the plasma membrane and the nucleus (FIG. 8) is described. First, this organelle harbours the small GTPase Rab5 but is distinguished from the canonical early endosomes as well as any established endocytic or biosynthetic organelles, by the presence of two Rab5 effectors: APPL1 and APPL2. Second, it is a specialised endosome displaying selectivity in cargo intemalisation. EGF but little transferrin and no fluid phase markers were intemalised into the APPL compartment, suggesting a specific role in signalling rather than housekeeping endocytosis. Third, following EGF intemalisation APPL1 is released from the membrane and translocates to the nucleus. Fourth, this release depends on the GTPase cycle of Rab5, an established regulator of endocytosis. Fifth, both APPL1 and APPL2 proteins interact with components of the nucleosome remodelling and histone deacetylase complex NuRD/MeCP1 and are required for cell proliferation. Sixth, deletion mutagenesis indicates that the interaction with Rab5 is essential for the regulation of proliferative activity of APPL1. Given its role in the transmission of signals, the APPL-positive compartment has been termed hermesome, after Hermes, the mythic messenger of Greek gods, and endosome. These findings have several important implications concerning the role of membrane compartmentalisation and endocytic transport in signal transduction.

EGF Signalling from Hermesomes

EGF uptake is traditionally a hallmark of Clathrin-, Dynamin- and Rab5-dependent endocytosis^{14,53}. The existence of a novel EGF entry route into hermesomes indicates that this view is incomplete. The fact that only a minor pool of EGF is intemalised into hermesomes, argues that the physical sequestration of EGF in this novel compartment may fulfil a signalling role rather than ligand-receptor downregulation and degradation. Importantly, the data presented in this application shed new light onto the seminal findings by Schmid and colleagues⁵³, who reported an enhancement of EGF-dependent proliferation in cells where Clathrin-mediated endocytosis was inhibited via the dominant negative Dynamin^{K44A} mutant. A residual EGF uptake (30% of the control) was observed under these conditions. The data presented here suggest that at least a fraction of this pool is most likely internalised into hermesomes. The data of Schmid and colleagues argue further that even if EGF signalling takes place on canonical early endosomes, it is dispensable for the mitogenic response. In contrast, the present inventors demonstrate that the interpretation of Schmid et al. does not take into account the hermesome pathway, and that APPL-dependent signalling pathways are required for cell proliferation, pointing to functional differences between signals emitted from hermesomes and canonical early endosomes. What is the intracellular fate of the hermesomal pool of EGF? According to the data accumulated in the present application, one may predict that at late time points after internalisation, EGF is cleared from hermesomes and joins the bulk of endocytosed ligand in conventional early and late endosomes, as previously described⁵⁴. The hermesome-associated pool of EGF may be routed to the canonical early endosomes Rab5-dependently, as expression of Rab5Q79L relocates APPL proteins to enlarged endosomes, suggesting a possible mixing of the two compartments (FIG. 1E).

Coupling GTPase Cycle of Rab5 to Signal Transduction

Remarkably, the studies performed in the course of solving the problem posed indicate that the cell utilises the simplest mechanism to couple the regulation of receptor trafficking to that of growth factor signalling: the shared GTPase switch of Rab5. The inventors established a model whereby such regulation is exploited both in time and space (FIG. 8). That is, Rab5 is present on at least four distinct intracellular compartments: plasma membrane, Clathrin-coated vesicles, early endosomes¹⁷⁻¹⁹ and hermesomes, where it recruits different sets of interacting proteins. This clearly implies that the correct targeting of effectors requires membrane-binding sites additional to Rab5⁵⁵. The physical separation of early endosomes and hermesomes provides the advantage of independent regulation of the Rab5 GTP/GDP cycle in response to growth factors as compared with a single organelle. In the canonical endocytic pathway, upon EGF stimulation Rab5 is activated at the plasma membrane and on early endosomes, allowing for efficient EGF intemalisation and downregulation^{12,14}. In contrast, EGF-induced release of APPL1 from hermesomes depends on the opposite effect on the Rab5 nucleotide cycle, i.e. stimulation of GTP hydrolysis. Subsequently, the level of Rab5-GTP must be re-equilibrated since APPL proteins return to hermesomes within 30 minutes of EGF stimulation. Interestingly, the established Rab5 GEF RIN1 and the GAP RN-tre are subjected to regulation by EGF¹²⁻¹⁴, but whether these or some yet uncharacterised family members account for the differential regulation of the Rab5 cycle on hermesomes will have to be determined. At least, it is evident that the kinetics of the Rab5 nucleotide cycle may also determine the residence time of EGF in hermesomes. In addition to restoring the localisation of APPL proteins, reactivation of Rab5 enables clearance of EGF by its further trafficking towards degradative compartments, thus allowing a new cycle of signalling. In this mechanism, Rab5 plays a dual role in regulating trafficking into/out of hermesomes and signalling from this compartment. Furthermore, spatial segregation between hermesomes and endosomes endows EGF with different temporal regulation and signal outputs.

Cellular Functions of APPL Proteins

The data compiled in the present application uncover for the first time a nucleo-cytoplasmic shuttling and an essential role of APPL proteins in the regulation of cell proliferation. By which mechanisms could APPL proteins exert this function? Two important clues were provided by the observations that APPL proteins translocate to the nucleus and, there, interact with the NuRD/MeCP1 complex. As histone deacetylase activities are required for cell cycle progression^{48,49}, APPL binding to NuRD/MeCP1 may serve the purpose of subjecting this function to regulation by extracellular signalling molecules. The inventors are not aware of any data linking the histone deacetylase/chromatin remodelling activities to endocytosis. Thus, their findings indicate the first example of such regulation. With the identification of APPL proteins as Rab5effectors the art is now in the position to explore this link further, a task that would be otherwise difficult to accomplish using Rab5 mutants, in view of their profound effects on the endocytic pathway and cellular homeostasis.

In summary, the present inventors have delineated a multi-step process (FIG. 8) in which 1) the interaction with Rab5 followed by 2) the release from hermesomes, 3) the import from cytoplasm to the nucleus and 4) the interaction with APPL effectors (i.e. molecules that act downstream APPL1/2) such as NuRD/MeCP1 as well as others to be identified constitute crucial steps of the cycle and are essential for the function of APPL1 in cell proliferation, these four steps reflecting the alternate options (i) to (iv) of the assay of screening for anti-cancer agents according to the invention, as described at page 4 of the description. The mutagenesis analysis implies that the Rab5-dependent localization and release of APPL1 from hermesomes regulate downstream cytoplasmic interactions that are required for transmitting proliferative signals. This conclusion is supported by the findings that all mutants unable to interact with Rab5 exerted dominant negative effects on DNA synthesis. These effects are most likely due to interference by the mutants with the activity of endogenous APPL1 through sequestration of cytoplasmic factors, as evidenced by the dominant negative phenotype of the Δ1-272 mutant, which is excluded from the nucleus. It further indicates that continuous rounds of binding of APPL1 to Rab5 and dissociation from hermesomes ensure the reversibility of such cytoplasmic interactions, otherwise permanently stabilised by the mutant proteins with irreparable effects on cell proliferation. Cycling through hermesomes may confer post-translational modifications to APPL proteins necessary to regulate their ability to interact with other partners. Signalling proteins often undergo a wide range of modifications which affect their intracellular localisation, pattern of interacting partners or stability, as exemplified by MAP kinases, p53 or Smad proteins^{1,56,57}. Although our data point at the nucleus as a primary site of APPL-NuRD/MeCP1 complex interactions, it cannot be excluded that some binding between APPL proteins and components of the complex may also take place in the cytoplasm, as significant cytoplasmic pools of PID/MTA2 and RbAp46 in addition to their nuclear localisation were observed, as also reported for PID/MTA2⁵⁸.

With the discovery of the interaction with Rab5 and the localisation to the hermesome, some of the earlier data on APPL1 will now have to be re-examined. Originally, APPL1 was shown to interact with the inactive form of the multifunctional anti-apoptotic kinase AKT2³⁰. Since inactive AKT kinases are predominantly cytosolic and their activation leading to translocation to the membrane requires PI3-K activity, it is unlikely that AKT2 colocalises with APPL proteins on hermesomes given their lack of the relevant phosphoinositides. Another reported interactor of APPL1 is the tumour suppressor DCC, a plasma membrane receptor for an axon-guiding molecule netrin-1^{31,59}. In the absence of ligand, DCC induces apoptosis via activation of caspase-3 and -9 in a process that requires APPL1^{31,60}. Neither the intracellular trafficking nor the ligand-dependence of the DCC-APPL1 interaction have been addressed, but an attractive possibility suggested by our work is that in neurons DCC could signal via hermesomes. Another exciting implication of our data concerns the possible link between APPL-mediated processes, such as DCC-induced apoptosis, to the action of p53, one of the substrates of NuRD/MeCP1. Activation of p53 induces either growth arrest or apoptosis, depending on the set of its transcriptional targets activated under various conditions⁶¹. In this context it appears particularly interesting that deacetylation of p53 mediated by a direct interaction with PID/MTA2 reduces its activity and apoptotic potential⁴⁷. Notably, the BAR domain of amphiphysinII/BIN1 has been shown to possess pro-apoptotic activity⁶² and we observed increased cell death upon over-expression of the BAR domain of APPL1 (FIG. 7C). Although we have not explored it further, the occasionally observed localisation of APPL1 to mitochondria may point to a role of the APPL proteins in apoptotic and stress responses.

The function of hermesomes is not restricted to the response to a single growth factor such as EGF. Rather, this organelle is responsible for the observed release of APPL1 from hermesomes upon oxidative stress. Likewise, growth factors other than EGF may be sorted into hermesomes in addition to early endosomes (as suggested by the interaction of APPL1 and DCC), and the resulting differences in the quality of generated signals are tightly regulated depending on the cell type or developmental stage, as it is known that the same growth factor can elicit either proliferation or differentiation response in various cells⁶³. The observed APPL/NuRD/MeCP1 interaction indicates that signalling via hermesomes is directly linked to chromatin remodelling, a process of crucial importance in development. This view is supported by recent studies demonstrating that the components of C. elegans NuRD are required for embryonic viability, patterning and Ras signalling^{50,64,65}. APPL proteins do not have homologues in C. elegans or Drosophila but are present in all vertebrates and play a signalling role during development, implied also by the interaction of APPL1 with DCC which functions in axon guidance³¹.

In summary, the identification of the hermesome as a new intracellular organelle acting as a platform for signalling and distinct from the canonical early endosomes—along with the existence of the hermesomes and the Rab5-dependent regulatory cycle of APPL proteins—has led to the possibility for therapeutic intervention based on anti-proliferative agents (as described in the instant application) without affecting the housekeeping functions of the canonical early endosomes.

### Methods

Protein Identification by Mass Spectrometry

Gel separated proteins were visualised by staining with Coomassie, excised from the gel slab and in-gel digested with trypsin as described⁶⁶. Tryptic peptides were sequenced by nanoelectrospray tandem mass spectrometry on hybrid quadrupole time-of-flight mass spectrometers Q-TOF I (Micromass Ltd, Manchester, UK) and QSTAR Pulsar i (MDS Sciex, Concord, Canada) as described in⁶⁷. Database searching was performed by Mascot software Matrix Science, Ltd, London).

APPL Cloning and Antibody Production

APPL1 and APPL2 were cloned from human full-length adult leukocyte cDNA library (Invitrogen Life Technologies) and by RT-PCR from HeLa mRNA, respectively. Peptides SSSQSEESDLGEGGKKRESEA+C and NDQPDDDDGNPNEHRGA+C derived from the sequence of APPL1 and APPL2, respectively, were synthesised and injected into rabbits (Eurogentec, Belgium). Sera were affinity purified using peptides immobilised on Sulfolink beads (Pierce).

Cell Culture, Transfections, Immunofluorescence, Immunoelectron Microscopy, Endosome Fusion Assay and BrdU Incorporation

HeLa, A431 and BHK cells were grown and immunofluorescence labelling were performed according to standard procedures. For transient expression studies, cells were transfected using FuGENE 6 (Roche) and analysed 20 h post-transfection. For immunoelectron microscopy cells were processed for frozen sections as described⁶⁸. BrdU incorporation was performed using Labeling and Detection Kit (Roche). Endosome fusion assay was performed as described⁴¹. Antibodies against PID/MTA2 and RbAp46 were obtained from Oncogene Research Products and Affinity Bioreagents, Inc, respectively.

siRNA Preparation and Transfection

Duplex siRNA (APPL1: 5′-CACACCUGACCUCAAAACUTT and 5′-AGUUUUGAGGUCAGGUGUGTT; APPL2: 5′-GUGGUGGAUGAGCUUAAUCTT and 5′-GAUUAAGCUCAUCCACCACTT) were purchased from Proligo (Paris, France) and transfected using Oligofectamine (Invitrogen).

Immunoprecipitation and GST Pulldown

HeLa cells grown in suspension (4 1) were pelleted, broken in the lysis buffer (50 mM Hepes pH 7.4, 150 mM KCl, 2 mM MgCl₂) by 10 passages through a cell cracker (EMBL, Heidelberg) and fractionated by centrifugation to obtain nuclei (4000×g) and cytosol (100 000×g). To produce total or nuclear detergent extracts, HeLa cells or nuclei were homogenised in the lysis buffer containing 1% Triton X-100, followed by 3 h solubilisation with rotation at 4° C. and centrifugation at 100 000×g to remove particulate material. For immunoprecipitations, antibodies were crosslinked with dimethyl pimelimidate (Pierce) to protein A agarose, incubated with extracts or cytosol at 4° C. overnight and washed extensively with the respective lysis buffers containing 500 mM KCl before elution with 100 mM glycine pH 2.5 (with 1% Triton X-100 in case of detergent extracts). For GST pulldown, glutathione-sepharose beads complexed with GST, GST-APPL1 and GST-APPL2 were incubated with nuclear extracts at 4° C. overnight, washed with the lysis buffer containing 1% Triton X-100 and eluted with the wash buffer supplemented with 25 mM glutathione. Fractions are analysed by Western blotting.

### EXAMPLES

### Example 1

Isolation of Hermesomes from Cultured Cells by Density Gradient Centrifugation

Two liters of S-HeLa cells are grown in suspension (in S-MEM containing 5% NCS, L-glutamine, non-essential amino acids and antibiotics) to the density of 0.8-1.2×10⁶ cells/ml. Cells are collected by centrifugation at 500 g for 10 min at 4° C., washed twice with PBS and resuspended in 2 cell volumes of ice cold SIM buffer (250 mM sucrose, 3 mM imidazole, 1 mM MgCl₂ pH 7.4) containing freshly added protease inhibitors and 1 mM DTT. Cells are broken by 7-10 passages through a ball-bearing homogeniser and the cell homogenate is spun in the tabletop centrifuge at 2500 g for 20 minutes at 4° C. to obtain post-nuclear supernatant (PNS). PNS is adjusted to 40.6% sucrose using the refractometer and an ice cold 62% stock solution of sucrose in 3 mM imidazole pH 7.4. Adjusted PNS is loaded at the bottom of 35-10% continuous gradient of sucrose in imidazole and centrifuged for 6 hours at 35,000 rpm in a Beckman SW40 rotor at 4° C. Fractions of 1 ml are collected, analysed for the presence of APPL proteins by Western blot and stored at −80° C. Fractions containing APPL 1 and/or 2 comprise hermesomes, the novel cell organelle according to the present invention.

### Example 2

Immunoisolation of Hermesomes from the Membrane Fraction of HeLa Cells

Inmunoisolation of hermesomes from the membrane fraction of HeLa cells is performed essentially as described by Trischler et al.⁶⁹ Briefly, affinity purified goat anti-rabbit IgGs are coupled to activated magnetic beads (p-toluene sulfonylchloride-activated Dynabeads M-450) according to the manufacturer's instructions (Dynal). Beads are incubated with anti-APPL1 affinity purified antibodies in PBS/0.5% bovine serum albumin (BSA) for 12 hours at 4° C., followed by three washes in PBS/0.5% BSA and 1 wash in PBS/0.1% BSA.

For immunoisolation, APPL1 antibody-coated magnetic beads are incubated with the hermesome-enriched fraction of S-HeLa membranes isolated on the sucrose gradient as described in Example 1 at a concentration of 60-80 mg protein/10 mg of beads on a rotating wheel for 4 hours at 4° C. Subsequently, beads with bound material are collected with a magnet and washed twice in PBS/0.1% BSA for 5 minutes each and once in PBS alone. Supernatants containing the non-bound material and an equal portion of the starting material are centrifuged at 100,000 g for 1 hour at 4° C. The samples are analysed by SDS-PAGE (12%) and immunoblotting.

### Example 3

In Vivo Assay for APPL-Mediated Signalling

Cells (primary cultures or established cell lines) are grown on coverslips, serum-starved for 12 h and treated with the compounds to be tested for various time periods. Subsequently, cells are incubated with either of the growth factors, fluorescently-labelled, as listed on page 6. Incubation was for 5-30 min at 37° C., followed by fixation with 3% paraformaldehyde, permeabilisation with 0.1% Triton X-100 and immunostaining with anti-APPL1 antibody, performed according to standard procedures. The degree of colocalisation of APPL1 and the growth factor, the solubilisation of APPL1 and its translocation to the nucleus are assessed by viewing the samples under the fluorescence microscope and quantifying the signals using the Metamorph program (Universal Imaging Corporation).

### Example 4

In Vitro Assay of Hermesome Function

Hermesomes isolated as described for Example 1 are analysed by quantitative Western blotting to assay the levels of Rab5, APPL1 and/or 2. To assess the abilities of hermesomes to recruit exogenous Rab5, reactions are set up on ice in a final volume of 60 μl, each reaction tube containing 15-20 μl hermesomes (isolated as described in Example 1), an ATP-regenerating system (freshly mixed 1:1:1 each: 4 mg/ml creatine kinase, 800 mM creatine phosphate and 100 mM ATP), and 1 mM GTP or GDP; in the absence or presence of 3 mg/ml cytosol, 100 nM Rab5-GDI complex, 4 μM RabGDI or the reagents to be tested. Reactions are incubated for 30 minutes at 37° C., diluted with 100 μl of ice-cold PBS and spun in a Beckman rotor TLA 100.2 at 70 000 rpm, 30 minutes at 4° C. Pellets are washed with 500 μl ice-cold PBS, recentrifuged for 5 min under the same conditions and resuspended in 60 μl SDS loading buffer by incubation for 20 min at 37° C. with shaking. Samples are analysed by SDS-PAGE and Western blotting for Rab5, APPL1/2 and other Rab5 effectors.

### REFERENCES

1. Chang, L. & Karin, M. Mammalian MAP kinase signalling cascades. Nature 410, 37-40. (2001).
2. Dumont, J. E., Dremier, S., Pirson, I. & Maenhaut, C. Cross signaling, cell specificity, and physiology. Am J Physiol Cell Physiol 283, C2-28. (2002).
3. Grimes, M. L. et al. Endocytosis of activated TrkA: evidence that nerve growth factor induces formation of signaling endosomes. J Neurosci 16, 7950-64. (1996).
4. Teis, D., Wunderlich, W. & Huber, L. A. Localization of the MP1-MAPK scaffold complex to endosomes is mediated by p14 and required for signal transduction. Dev Cell 3, 803-14. (2002).
5. McPherson, P. S., Kay, B. K. & Hussain, N. K. Signaling on the endocytic pathway. Traffic 2, 375-84. (2001).
6. Di Fiore, P. P. & De Camilli, P. Endocytosis and Signaling: An Inseparable Partnership. Cell 106, 1-4 (2001).
7. Sorkin, A. & Von Zastrow, M. Signal transduction and endocytosis: close encounters of many kinds. Nat Rev Mol Cell Biol 3, 600-14. (2002).
8. Lu, Z. et al. Transforming growth factor beta activates Smad2 in the absence of receptor endocytosis. J Biol Chem 277, 29363-8. (2002).
9. Johannessen, L. E., Ringerike, T., Molnes, J. & Madshus, I. H. Epidermal growth factor receptor efficiently activates mitogen-activated protein kinase in HeLa cells and Hep2 cells conditionally defective in clathrin-dependent endocytosis. Exp Cell Res 260, 136-45. (2000).
10. Kao, A. W., Ceresa, B. P., Santeler, S. R. & Pessin, J. E. Expression of a dominant interfering dynamin mutant in 3T3L1 adipocytes inhibits GLUT4 endocytosis without affecting insulin signaling. J Biol Chem 273, 25450-7. (1998).
11. Mellman, I. Endocytosis and molecular sorting. Annu Rev Cell Dev Biol 12, 575-625 (1996).
12. Barbieri, M. A. et al. Epidermal growth factor and membrane trafficking. EGF receptor activation of endocytosis requires Rab5a. J Cell Biol 151, 539-50. (2000).
13. Tall, G. G., Barbieri, M. A., Stahl, P. D. & Horazdovsky, B. F. Ras-Activated Endocytosis Is Mediated by the Rab5 Guanine Nucleotide Exchange Activity of RIN1. Dev Cell 1, 73-82 (2001).
14. Lanzetti, L. et al. The Eps8 protein coordinates EGF receptor signalling through Rac and trafficking through Rab5. Nature 408, 374-7. (2000).
15. Rybin, V. et al. GTPase activity of rab5 acts as a timer for endocytic membrane fusion. Nature 383, 266-269 (1996).
16. Martinu, L., Santiago-Walker, A., Qi, H. & Chou, M. M. Endocytosis of epidermal growth factor receptor regulated by Grb2-mediated recruitment of the Rab5 GAP RN-tre. J Biol Chem 277, 50996-51002 (2002).
17. Bucci, C. et al. The small GTPase rab5 functions as a regulatory factor in the early endocytic pathway. Cell 70, 715-728 (1992).
18. Rubino, M., Miaczynska; M., Lippe, R. & Zerial, M. Selective membrane recruitment of EEA1 suggests a role in directional transport of clathrin-coated vesicles to early endosomes. J Biol Chem 275, 3745-8 (2000).
19. Christoforidis, S., McBride, H. M., Burgoyne, R, D. & Zerial, M. The Rab5 effector EEA1 is a core component of endosome docking. Nature 397, 621-625 (1999).
20. Simonsen, A. et al. EEA1 links phosphatidylinositol 3-kinase function to Rab5 regulation of endosome fusion. Nature 394, 494-498 (1998).
21. Christoforidis, S. et al Phosphoinositide-3-Kinases are Rab5 effectors. Nat Cell Biol 1, 249-252 (1999).
22. Nielsen, E. et al. Rabenosyn-5, a novel Rab5 effector, is complexed with hVPS45 and recruited to endosomes through a FYVE finger domain. J Cell Biol 151, 601-12. (2000).
23. Lippe, R., Miaczynska, M., Rybin, V., Runge, A. & Zerial, M. Functional Synergy between Rab5 Effector Rabaptin-5 and Exchange Factor Rabex-5 When Physically Associated in a Complex. Mol Biol Cell 12, 2219-28. (2001).
24. Zerial, M. & McBride, H. Rab proteins as membrane organizers. Nat Rev Mol Cell Biol 2, 107-17. (2001).
25. Tsukazaki, T., Chiang, T. A., Davison, A. F., Attisano, L. & Wrana, J. L. SARA, a FYVE domain protein that recruits Smad2 to the TGFbeta receptor. Cell 95, 779-91. (1998).
26. Itoh, F. et al. The FYVE domain in Smad anchor for receptor activation (SARA) is sufficient for localization of SARA in early endosomes and regulates TGF-beta/Smad signalling. Genes Cells 7, 321-31. (2002).
27. Komada, M. & Kitamura, N. Growth factor-induced tyrosine phosphorylation of Hrs, a novel 115-kilodalton protein with a structurally conserved putative zinc finger domain. Mol Cell Biol 15, 6213-21. (1995).
28. Raiborg, C. et al. FYVE and coiled-coil domains determine the specific localisation of Hrs to early endosomes. J Cell Sci 114, 2255-63. (2001).
29. Panopoulou, E. et al. Early endosomal regulation of Smad-dependent signaling in endothelial cells. J Biol Chem 277, 18046-52. (2002).
30. Mitsuuchi, Y. et al. Identification of a chromosome 3p14.3-21.1 gene, APPL, encoding an adaptor molecule that interacts with the oncoprotein-serine/threonine kinase AKT2. Oncogene 18, 4891-8. (1999).
31. Liu, J. et al. Mediation of the DCC apoptotic signal by DIP13 alpha. J Biol Chem 277, 26281-5. (2002).
32. Nakai, K. & Horton, P. PSORT: a program for detecting sorting signals in proteins and predicting their subcellular localization. Trends Biochem Sci 24, 34-6. (1999).
33. Schultz, J., Milpetz, F., Bork, P. & Ponting, C. P. SMART, a simple modular architecture research tool: identification of signaling domains. Proc Natl Acad Sci USA 95, 5857-64. (1998).
34. Sakamuro, D., Elliott, K. J., Wechsler-Reya, R. & Prendergast, G. C. BIN1 is a novel MYC-interacting protein with features of a tumour suppressor. Nat Genet 14, 69-77. (1996).
35. Altschul, S. F. et al. Gapped BLAST and PSI-BLAST: a new generation of protein database search programs. Nucleic Acids Res 25, 3389-402. (1997).
36. Fischer, D. et al. CAFASP-1: critical assessment of fully automated structure prediction methods. Proteins Suppl, 209-17. (1999).
37. Van Aelst, L., Joneson, T. & Bar-Sagi, D. Identification of a novel Rac1-interacting protein involved in membrane ruffling. EMBO J 15, 3778-86. (1996).
38. Tarricone, C. et al. The structural basis of Arfaptin-mediated cross-talk between Rac and Arf signalling pathways. Nature 411, 215-9. (2001).
39. Stenmark, H. et al. Inhibition of rab5 GTPase activity stimulates membrane fusion in endocytosis. EMBO J. 13, 1287-1296 (1994).
40. Sonnichsen, B., De Renzis, S., Nielsen, E., Rietdorf, J. & Zerial, M. Distinct membrane domains on endosomes in the recycling pathway visualized by multicolor imaging of Rab4, Rab5, and Rab11. J Cell Biol 149, 901-14. (2000).
41. Horiuchi, H. et al. A novel Rab5 GDP/GTP exchange factor complexed to Rabaptin-5 links nucleotide exchange to effector recruitment and function. Cell 90, 1149-1159 (1997).
42. Patki, V. et al. Identification of an early endosomal protein regulated by phosphatidylinositol 3-kinase. Proc Natl Acad Sci USA 94, 7326-30 (1997).
43. Pelkmans, L., Kartenbeck, J. & Helenius, A. Caveolar endocytosis of simian virus 40 reveals a new two-step vesicular-transport pathway to the ER. Nat Cell Biol 3, 473-83. (2001).
44. Sabharanjak, S., Sharma, P., Parton, R. G. & Mayor, S. GPI-anchored proteins are delivered to recycling endosomes via a distinct cdc42-regulated, clathrin-independent pinocytic pathway. Dev Cell 2, 411-23. (2002).
45. Cavalli, V. et al. The stress-induced MAP kinase p38 regulates endocytic trafficking via the GDI:Rab5 complex. Mol Cell 7, 421-32. (2001).
46. Feng, Q. & Zhang, Y. The MeCP1 complex represses transcription through preferential binding, remodeling, and deacetylating methylated nucleosomes. Genes Dev 15, 827-32. (2001).
47. Luo, J., Su, F., Chen, D., Shiloh, A. & Gu, W. Deacetylation of p53 modulates its effect on cell growth and apoptosis. Nature 408, 377-81. (2000).
48. Yoshida, M., Horinouchi, S. & Beppu, T. Trichostatin A and trapoxin: novel chemical probes for the role of histone acetylation in chromatin structure and function. Bioessays 17, 423-30. (1995).
49. Taunton, J., Hassig, C. A. & Schreiber, S. L. A mammalian histone deacetylase related to the yeast transcriptional regulator Rpd3p. Science 272, 408-11. (1996).
50. Ahringer, J. NuRD and SIN3 histone deacetylase complexes in development. Trends Genet 16, 351-6. (2000).
51. Elbashir, S. M. et al. Duplexes of 21-nucleotide RNAs mediate RNA interference in cultured mammalian cells. Nature 411, 494-8. (2001).
52. Dowler, S. et al. Identification of pleckstrin-homology-domain-containing proteins with novel phosphoinositide-binding specificities. Biochein J 351, 19-31. (2000).
53. Vieira, A. V., Lamaze, C. & Schmid, S. L. Control of EGF receptor signaling by clathrin-mediated endocytosis. Science 274, 2086-9 (1996).
54. Jäckle, S., Runquist, E. A., Miranda-Brady, S. & Havel, R. J. Trafficking of the epidermal growth factor receptor and transferrin in three hepatocytic endosomal fractions. J Biol Chem 266, 1396-1402 (1991).
55. Miaczynska, M. & Zerial, M. Mosaic organization of the endocytic pathway. Exp Cell Res 272, 8-14. (2002).
56. Giaccia, A. J. & Kastan, M. B. The complexity of p53 modulation: emerging patterns from divergent signals. Genes Dev 12, 2973-83. (1998).
57. Gronroos, E., Hellman, U., Heldin, C. H. & Ericsson, J. Control of Smad7 stability by competition between acetylation and ubiquitination. Mol Cell 10, 483-93. (2002).
58. Humphrey, G. W. et al. Stable histone deacetylase complexes distinguished by the presence of SANT domain proteins CoREST/kiaa0071 and Mta-L1. J Biol Chem 276, 6817-24. (2001).
59. Keino-Masu, K. et al. Deleted in Colorectal Cancer (DCC) encodes a netrin receptor. Cell 87, 175-85. (1996).
60. Forcet, C. et al. The dependence receptor DCC (deleted in colorectal cancer) defines an alternative mechanism for caspase activation. Proc Natl Acad Sci USA 98, 3416-21. (2001).
61. Vousden, K. H. p53: death star. Cell 103, 691-4. (2000).
62. Elliott, K., Ge, K., Du, W. & Prendergast, G. C. The c-Myc-interacting adaptor protein Bin1 activates a caspase-independent cell death program. Oncogene 19, 4669-84. (2000).
63. Yarden, Y. The EGFR family and its ligands in human cancer. signalling mechanisms and therapeutic opportunities. Eur J Cancer 37 Suppl 4, S3-8. (2001).
64. Unhavaithaya, Y. et al. MEP-1 and a Homolog of the NURD Complex Component Mi-2 Act Together to Maintain Germline-Soma Distinctions in C. elegans. Cell 111, 991-1002. (2002).
65. Lu, X. & Horvitz, H. R. lin-35 and lin-53, two genes that antagonize a C. elegans Ras pathway, encode proteins similar to Rb and its binding protein RbAp48. Cell 95, 981-91. (1998).
66. Shevchenko, A., Wilm, M., Vorm, O. & Mann, M. Mass Spectrometric Sequencing of Proteins from Silver Stained Polyacrylamide Gels. Anal Chem 68, 850-858 (1996).
67. Shevchenko, A. et al. Rapid ‘de Novo’ Peptide Sequencing by a Combination of Nanoelectrospray, Isotopic Labeling and a Quadrupole/Time-of flight Mass Spectrometer. Rapid Comm Mass Spectrom 11, 1015-1024 (1997).
68. Luetterforst, R. et al. Molecular characterization of caveolin association with the Golgi complex: identification of a cis-Golgi targeting domain in the caveolin molecule. J Cell Biol 145, 1443-59 (1999).
69. Trischler et al. Biochemical analysis of distinct Rab5- and Rab11-positive endosomes along the transferrin pathway. J Cell Sci. 112, 4773-83 1999).

1. An method of screening for an anti-proliferative drug comprising the steps of:
(a) contacting cells of a primary cell culture or of an established cell line with a candidate substance,
(b) subsequently or concomitantly with contacting of the candidate substance, contacting the cells with a growth factor;
(c) processing the cells for immunofluorescence staining to detect APPL1 and APPL2 using an anti-APPL1 and/or 2 antibody, or alternatively using GFP-tagged APPL proteins stably or transiently expressed by the cells via transfection;
(d) assessing the degree of colocalisation of APPL1 and/or 2 and the growth factor, the solubilisation of APPL1 and/or 2 and their translocation to the nucleus;
(e) repeating steps (b) to (d) with cells not previously treated with the candidate substance; and
(f) comparing the degree of colocalisation of APPL1 and/or 2 and the growth factor, the solubilisation of APPL1 and/or 2 and their translocation to the nucleus between the cells not previously treated with the candidate substance (untreated cells) and cells treated with the candidate substance (treated cells),
wherein an altered degree of colocalisation of APPL1 and/or 2 and the growth factor, an altered solubilisation of APPL1 and/or 2 and/or their altered translocation to the nucleus in the treated vs. the untreated cells identifies the candidate substance as an anti-proliferative drug.

2. The assay of claim 1, wherein the growth factor is an epidermal growth factor (EGF) family, a fibroblast growth factor (FGF), a transforming growth factor-β (TGFs-β), a transforming growth factor-α (TGF-α), an insulin-like growth factor such as IGF-I and IGF-II, a tumour necrosis factor such as TNF-α and TNF-α, a vascular endothelial growth factor (VEGF), a nerve growth factor (NGF), a hepatocyte growth factor/scatter factor, pleiotrophin, oncostatin M (OSM), an angiogenic factor (angiogenin), an ephrin, an interleukin (IL) such as IL1-13, an interferon (INF) such as IFN-α,-β,-γ, a colony stimulating factor (CSF), erythropoietin (EPO), or a platelet-derived growth factor (PDGF).

3. The assay of claim 1, wherein the growth factor and/or the antibody are/is labelled, preferably by fluorescence, and/or wherein step (d) of assessing (i) the degree of colocalisation, (ii) the solubilisation and (iii) the translocation is performed by fluorescence microscopy.

4. An anti-proliferative drug identified and/or isolated according to the method of claim 1.

5. A method of treating a cancer/tumour disease comprising contacting a subject with a cancer/tumour disease with an anti-proliferative drug identified and/or isolated according to the method of claim 1.

6. The method of claim 5, wherein the treatment occurs by an inhibition of proliferation and/or induction of apoptosis in cancer/tumour cells.

7. An method of screening for an anti-proliferative drug, comprising the steps of:
(a) isolating hermosomes from cells of a cell culture, in particular by density gradient centrifugation;
(b) restoring their functionality by contacting the hermesomes with cytosol, an ATP-regenerating system and either or both of GTP and GDP;
(c) modulating their function in cell proliferation and/or apoptosis by contacting with a candidate substance; and
(d) comparing the hernesomes isolated from cells previously treated with or without the growth factor (stimulated or non-stimulated cells), with or without the candidate substance (treated or untreated cells) or exposed to a candidate substance after isolation.

8. The method of claim 7, wherein the candidate substance modulates (i) the recruitment of Rab5 on hermesome, (ii) the activity of Rab5 and the release of APPL1and/or APPL2 from hermesomes, and/or (iii) the ability of the released APPL proteins to interact with the NuRD/MeCP1 complex or its associated factors such as p53.
